(19) United States
(12) Patent Application Publication
(10) Pub. No.: US 2024/0115151 A1
(43) Pub. Date: Apr. 11, 2024

(54) PHYSIOLOGICAL SIGNAL MEASUREMENT SYSTEM, PHYSIOLOGICAL SIGNAL MEASUREMENT METHOD, AND MOBILE DEVICE PROTECTIVE CASE

(71) Applicant: INDUSTRIAL TECHNOLOGY RESEARCH INSTITUTE, Hsinchu (TW)

(72) Inventors: Yun-Yi HUANG, Hsinchu City (TW); Yu-Chiao TSAI, Hsinchu City (TW); Chun LIU, Hsinchu City (TW); Heng-Yin CHEN, Hsinchu City (TW)

(73) Assignee: INDUSTRIAL TECHNOLOGY RESEARCH INSTITUTE, Hsinchu (TW)

(21) Appl. No.: 18/377,063

(22) Filed: Oct. 5, 2023

Related U.S. Application Data
(60) Provisional application No. 63/413,294, filed on Oct. 5, 2022.

(51) Int. Cl. A61B 5/053; A61B 5/00

(52) U.S. Cl. CPC A61B 5/053; A61B 5/681; A61B 5/6898; A61B 5/7225; A61B 5/6891; A61B 5/6893; A61B 5/748; A61B 2560/0223

(57) ABSTRACT

A physiological signal measurement system, a physiological signal measurement method, and a mobile device protective case are provided. The physiological signal measurement system includes a first electrode, a second electrode, a reference electrode, an impedance front-end circuit module and a dynamic signal matching module. The first electrode, the second electrode and the reference electrode are used to obtain a first sensing signal and a second sensing signal. The impedance front-end circuit module is used to detect a first impedance of the first electrode and a second impedance of the second electrode, and obtain an original differential signal according to the first sensing signal and the second sensing signal. The dynamic signal matching module is used to obtain a calibration sequence according to the first impedance, the second impedance and the original differential signal, and obtain a compensated calibration sequence according to the calibration sequence and the original differential signal.

This application claims the benefit of U.S. Provisional application Ser. No. 63/413,294, filed Oct. 5, 2022, the disclosure of which is incorporated by reference herein in its entirety.

### TECHNICAL FIELD

The disclosure relates in general to a signal measurement system, a signal measurement method and a protective case, and also relates to a physiological signal measurement system, a physiological signal measurement method and a mobile device protective case.

### BACKGROUND

In order to achieve good health management, the physiological signal measurement technology is gradually applied for the portable electronic devices or the wearable devices. However, the implementation of the physiological signal measurement technology in the portable electronic devices or the wearable devices will face the problem of plastic materials with high insulation resistance or the large impedance difference between different materials without producing the coupling capacitance effect.

If the portable electronic device or the wearable device are used to implement two-hand or one-hand measurement, the materials at the contact points will be quite different, which will cause the differential signal to vary too much, making measurement impossible.

### SUMMARY

The disclosure is directed to a physiological signal measurement system, a physiological signal measurement method and a mobile device protective case. The impedance front-end circuit technology and the dynamic signal matching technology are used. Even if the materials of the contact points for the electrodes are quite different, or the insulating materials are used as the contact points, the measurement can still be carried out and correct physiological signals can be obtained after compensation. In this way, the electrodes could be disposed at various positions of the electronic device. Each position of the electronic device could be used to dispose the electrode. Thus, one-hand measurement or two-hand measurement could be achieved on various electronic devices according to the user's usual usage habits.

According to one embodiment, a physiological signal measurement system is proposed. The physiological signal measurement system includes a first electrode, a second electrode, a reference electrode, an impedance front-end circuit module and a dynamic signal matching module. The first electrode and the reference electrode are used to obtain a first sensing signal. The second electrode and the reference electrode are used to obtain a second sensing signal. The impedance front-end circuit module is used to detect a first impedance of the first electrode and a second impedance of the second electrode, and obtain an original differential signal according to the first sensing signal and the second sensing signal. The dynamic signal matching module is used to obtain a calibration sequence according to the first impedance, the second impedance and the original differential signal, and obtain a compensated calibration sequence according to the calibration sequence and the original differential signal.

According to another embodiment, a physiological signal measurement method is provided. The physiological signal measurement method includes the following steps: detecting a first impedance of the first electrode; detecting a second impedance of the second electrode; obtaining a first sensing signal; obtaining a second sensing signal; obtaining an original differential signal according to the first sensing signal and the second sensing signal; obtaining a calibration sequence according to the first impedance, the second impedance and the original differential signal; and obtaining a compensated calibration sequence according to the calibration sequence and the original differential signal.

According to an alternative embodiment, a mobile device protective case is provided. The mobile device protective case includes a case, a first electrode, a second electrode, a reference electrode and a processing system. The first electrode disposed at an inner side of the case. The second electrode disposed at the inner side of the case. The reference electrode disposed at the inner side of the case. The first electrode and the reference electrode are used to obtain a first sensing signal. The second electrode and the reference electrode are used to obtain a second sensing signal. The processing system is disposed at the inner side of the case. The processing system is coupled to the first electrode, the second electrode and the reference electrode. The processing system includes an impedance front-end circuit module and a dynamic signal matching module. The impedance front-end circuit module is configured to detect a first impedance of the first electrode and a second impedance of the second electrode, and obtain an original differential signal according to the first sensing signal and the second sensing signal. The dynamic signal matching module is configured to obtain a calibration sequence according to the first impedance, the second impedance and the original differential signal, and obtain a compensated calibration sequence according to the calibration sequence and the original differential signal.

### BRIEF DESCRIPTION OF THE DRAWINGS

FIG. 1A shows a diagram of a mobile device capable of measuring physiological signals according to one embodiment.

FIG. 1B shows a side perspective view of the mobile device of the FIG. 1A.

FIG. 2 shows a block diagram of a physiological signal measurement system according to one embodiment.

FIG. 3 shows a detailed block diagram of a coupling circuit according to one embodiment.

FIG. 4 shows a flow chart of a physiological signal measurement method according to one embodiment.

FIG. 5 illustrates processing in the steps of the physiological signal measurement method.

FIG. 6A shows a schematic diagram of a mobile device capable of measuring physiological signals according to an embodiment.

FIG. 6B shows a side perspective view of the mobile device of the FIG. 6A.

FIG. 7A shows a schematic diagram of a mobile device capable of measuring physiological signals according to another embodiment.

FIG. 7B shows a side perspective view of the mobile device of the FIG. 7A.

FIG. 8 shows a schematic diagram of a mobile device capable of measuring physiological signals according to another embodiment.

FIG. 9A shows a schematic diagram of a mobile device capable of measuring physiological signals according to another embodiment.

FIG. 9B shows a side perspective view of the mobile device of the FIG. 9A.

FIG. 10A shows a schematic diagram of a mobile device capable of measuring physiological signals according to another embodiment.

FIG. 10B shows a side perspective view of the mobile device of the FIG. 10A.

FIG. 11A shows a schematic diagram of a mobile device protective case capable of measuring physiological signals according to another embodiment.

FIG. 11B shows a side perspective view of the mobile device protective case of the FIG. 11A and a mobile device.

FIG. 12A shows a schematic diagram of a laptop capable of measuring physiological signals according to an embodiment.

FIG. 12B shows a side perspective view of the laptop of the FIG. 12A.

FIG. 13A shows a schematic diagram of a laptop capable of measuring physiological signals according to another embodiment.

FIG. 13B shows a side perspective view of the laptop of the FIG. 13A.

FIG. 14 shows a schematic diagram of a touchpad capable of measuring physiological signals according to an embodiment.

FIG. 15 shows a schematic diagram of a keyboard capable of measuring physiological signals according to an embodiment.

FIG. 16 shows a schematic diagram of a mouse capable of measuring physiological signals according to an embodiment.

FIG. 17 shows a schematic diagram of a mouse capable of measuring physiological signals according to another embodiment.

FIG. 18 shows a schematic diagram of a smart watch capable of measuring physiological signals according to an embodiment.

FIG. 19 shows a schematic diagram of a smart watch capable of measuring physiological signals according to another embodiment.

FIG. 20 shows a schematic diagram of a smart watch capable of measuring physiological signals according to another embodiment.

FIG. 21 shows a block diagram of a physiological signal measurement system according to another embodiment.

FIG. 22 shows a schematic diagram of a steering wheel capable of measuring physiological signals according to an embodiment.

FIG. 23A illustrates a front view of a steering wheel capable of measuring physiological signals according to another embodiment.

FIG. 23B illustrates a back view of the steering wheel capable of measuring physiological signals according to another embodiment.

FIG. 24 illustrates a schematic diagram of a seat capable of measuring physiological signals according to an embodiment.

FIG. 25A shows a schematic diagram of the left side of a gear lever capable of measuring physiological signals according to an embodiment.

FIG. 25B shows a schematic diagram of the right side of the gear lever capable of measuring physiological signals according to an embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

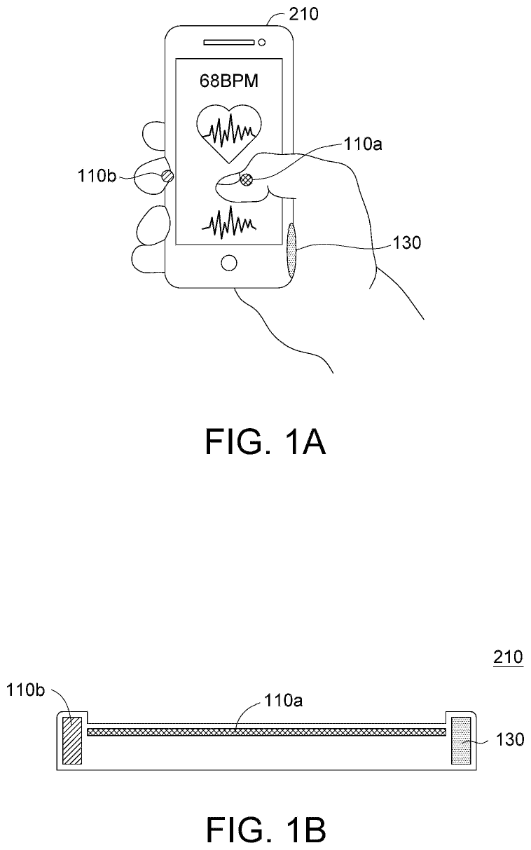

Please refer to FIG. 1A. FIG. 1A shows a diagram of a mobile device 210 capable of measuring physiological signals according to one embodiment. The physiological signal is, for example, an Electrocardiography (ECG) signal or an Electromyography (EMG) signal. The mobile device 210 capable of measuring the physiological signals is, for example, a mobile phone, a tablet computer, a laptop, an e-reader or a handheld game console. The mobile device 210 capable of measuring the physiological signals includes a first electrode 110a, a second electrode 110b and a reference electrode 130. The first electrode 110a, the second electrode 110b and the reference electrode 130 are, for example, a sheet-like structure or a point-like structure. The first electrode 110a could be disposed on a screen glass plate, a side frame or a back plate of the mobile device 210. The second electrode 110b could be disposed on the screen glass plate, the side frame or the back plate of the mobile device 210. The reference electrode 130 could be disposed at the screen glass plate, the side frame or the back plate of the mobile device 210. If the first electrode 110a, the second electrode 110b and the reference electrode 130 are arranged adjacent to each other, they are isolated from each other.

In the embodiment of the FIG. 1A, the first electrode 110a is disposed on the screen glass plate of the mobile device 210, the second electrode 110b is disposed on the side frame of the mobile device 210, and the reference electrode 130 is disposed on another side frame of the mobile device 210. As shown in the FIG. 1A, the user could hold the mobile device 210 with one hand. The thumb of the user's one hand could touch the screen glass plate provided with the first electrode 110a. The middle finger of the user's same hand could touch the side frame provided with the second electrode 110b. The inner side of the palm of the user's same hand could touch the side frame provided with the reference electrode 130. In this way, the one-hand measurement for the physiological signals could be realized on the mobile device 210.

Please refer to FIG. 1B. FIG. 1B shows a side perspective view of the mobile device 210 of the FIG. 1A. The second electrode 110b and the reference electrode 130 are disposed at the inner sides of the two opposite side frames of the mobile device 210. The first electrode 110a is disposed at the inner side of the screen glass plate of the mobile device 210. That is to say, when measuring the physiological signals, the user's fingers/palm and the first electrode 110a, the second electrode 110b and the reference electrode 130 will be separated by the screen glass plate or the side frames. The materials of the side frames are, for example, a high-impedance plastic or a low-impedance alloy. All of the screen glass plate and the plastic side frames may have high impedance, or the impedance difference between the screen glass plate and the alloy side frames may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to solve the above problems. Detailed technical content is described below.

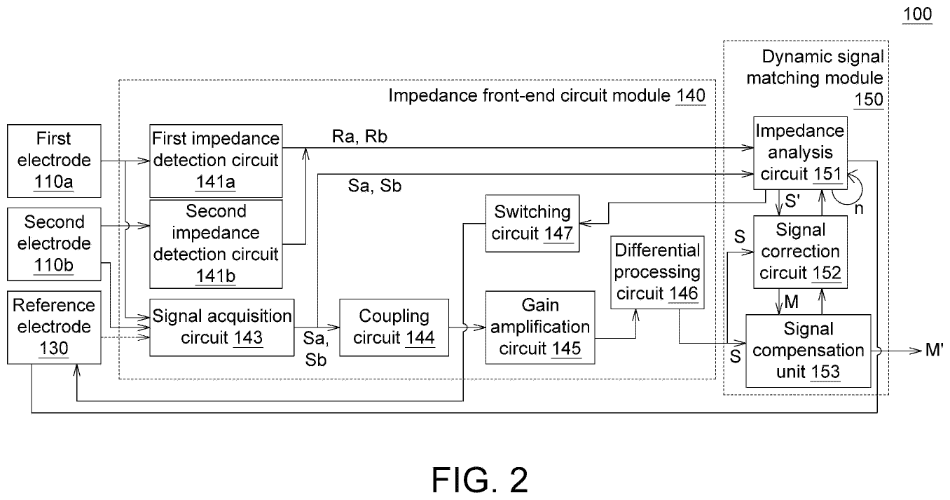

Please refer to FIG. 2. FIG. 2 shows a block diagram of the physiological signal measurement system 100 according to one embodiment. The physiological signal measurement system 100 includes the first electrode 110a, the second electrode 110b, the reference electrode 130, an impedance front-end circuit module 140 and a dynamic signal matching module 150. The first electrode 110a and the reference electrode 130 are used to obtain a first sensing signal Sa. The second electrode 110b and the reference electrode 130 are used to obtain a second sensing signal Sb.

The impedance front-end circuit module 140 includes a first impedance detection circuit 141a, a second impedance detection circuit 141b, a signal acquisition circuit 143, a coupling circuit 144, a gain amplification circuit 145, a differential processing circuit 146 and a switching circuit 147. The first impedance detection circuit 141a is connected to the first electrode 110a. The second impedance detection circuit 141b is connected to the second electrode 110b. The signal acquisition circuit 143 is connected to the first electrode 110a, the second electrode 110b and the reference electrode 130. The coupling circuit 144 connected to the signal acquisition circuit 143. The gain amplification circuit 145 is connected to the coupling circuit 144. The differential processing circuit 146 is connected to the gain amplification circuit 145.

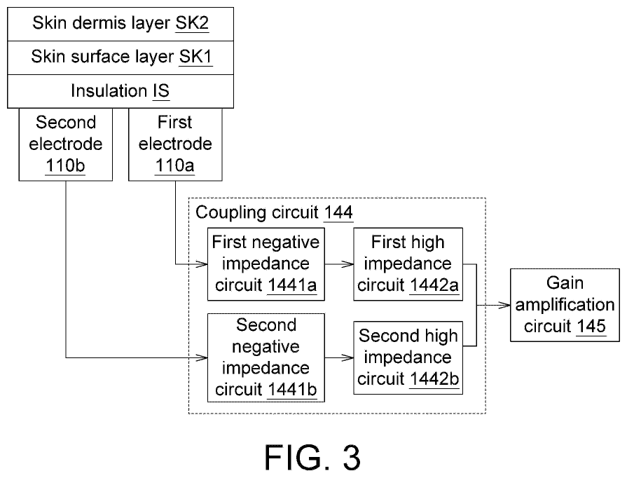

Please refer to FIG. 3. FIG. 3 shows a detailed block diagram of the coupling circuit 144 according to one embodiment. The coupling circuit 144 includes a first negative impedance circuit 1441a, a first high impedance circuit 1442a, a second negative impedance circuit 1441b and a second high impedance circuit 1442b. The first negative impedance circuit 1441a is connected to the first electrode 110a. The first high impedance circuit 1442a is connected to the first negative impedance circuit 1441a. The second negative impedance circuit 1441b is connected to the second electrode 110b. The second high impedance circuit 1442b is connected to the second negative impedance circuit 1441b. The first electrode 110a and the second electrode 110b detect a skin surface layer SK1 and a skin dermis layer SK2 through an insulation IS. In this embodiment, through the design of the first negative impedance circuit 1441a, the first high impedance circuit 1442a, the second negative impedance circuit 1441b and the second high impedance circuit 1442b, the noise interference could be reduced, the signal-to-noise ratio (SNR) could be improved, the input impedance frequency response could be improved, and the common-mode voltage could be reduced.

As shown in the FIG. 2, the dynamic signal matching module 150 includes an impedance analysis circuit 151, a signal correction circuit 152 and a signal compensation unit 153. The signal compensation unit 153 is, for example, a circuit, a chip, a circuit board, or a storage device for storing program code. The impedance analysis circuit 151 is connected to the first impedance detection circuit 141a and the second impedance detection circuit 141b. The signal correction circuit 152 is connected to the impedance analysis circuit 151 and the differential processing circuit 146. The signal compensation unit 153 is connected to the signal correction circuit 152 and the differential processing circuit 146.

In this embodiment, the impedance or signal is detected and converted through the components of the impedance front-end circuit module 140. Then, the signal could be corrected and compensated through the components of the dynamic signal matching module 150. The problems of inability to produce coupling capacitance effect and excessive differential signal variation caused by too high impedance and too high impedance difference could be effectively solved. The following is a flowchart to explain the operation of the above components.

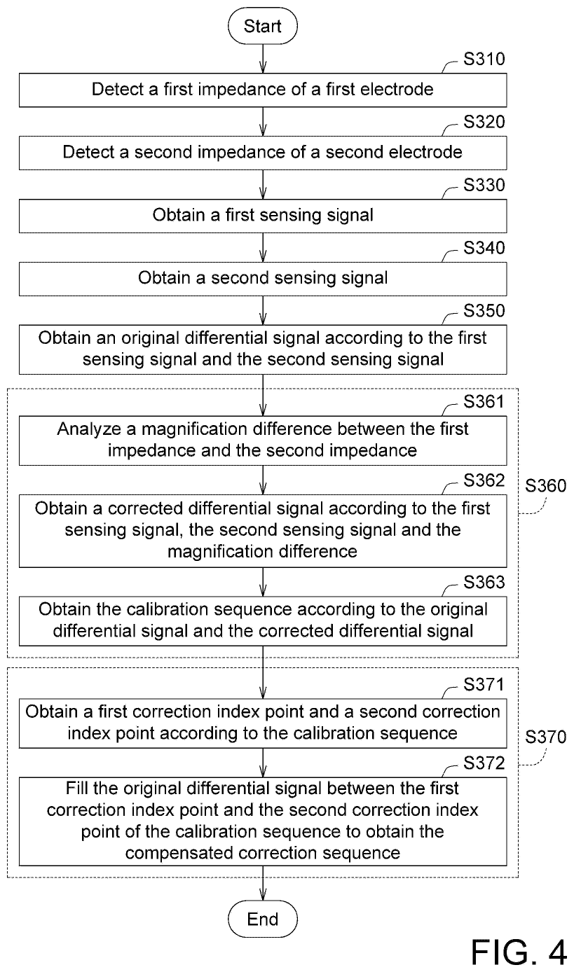

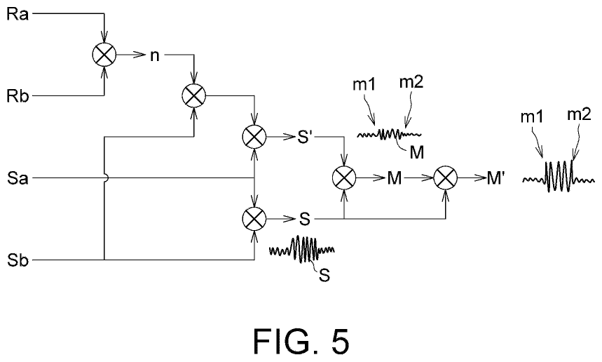

Please refer to FIG. 4 and FIG. 5. FIG. 4 shows a flow chart of the physiological signal measurement method according to one embodiment. FIG. 5 illustrates processing in the steps of the physiological signal measurement method. In step S310, the first impedance detection circuit 141a detects a first impedance Ra of the first electrode 110a.

In step S320, the second impedance detection circuit 141b detects a second impedance Rb of the second electrode 110b.

In step S330, the signal acquisition circuit 143 obtains the first sensing signal Sa through the first electrode 110a and the reference electrode 130.

In step S340, the signal acquisition circuit 143 obtains the second sensing signal Sb through the second electrode 110b and the reference electrode 130. The order of the above-mentioned steps S310 to S340 is not intended to limit the present disclosure. For example, steps S310 to S340 could be executed at the same time. When the difference between the impedances detected by the first impedance detection circuit 141a and the second impedance detection circuit 141b are greater than a predetermined value, the reference electrode 130 is turned on by the switching circuit 147.

Next, in step S350, the coupling circuit 144, the gain amplification circuit 145 and the differential processing circuit 146 obtain an original differential signal S according to the first sensing signal Sa and the second sensing signal Sb. In this step, the coupling circuit 144 is used to improve the signal-to-noise ratio of the first sensing signal Sa and the second sensing signal Sb. Next, the gain amplification circuit 145 is used to amplify the first sensing signal Sa and the second sensing signal Sb. Then, the differential processing circuit 146 is used to obtain the original differential signal S. As shown in the FIG. 5, the original differential signal S may include a high-amplitude section generated by the physiological signal and a low-amplitude section generated by the noise. In some conditions, it is difficult to distinguish between the high-amplitude section and the low-amplitude section, which will affect the measurement accuracy of the physiological signal. In this embodiment, compensation and correction could be performed through the following steps to improve the measurement accuracy for the physiological signals.

Then, in step S360, as shown in FIG. 5, the dynamic signal matching module 150 obtains a calibration sequence M according to the first impedance Ra, the second impedance Rb and the original differential signal S. The step S360 includes steps S361 to S363.

In the step S361, as shown in the FIG. 5, the impedance analysis circuit 151 of the dynamic signal matching module 150 analyzes a magnification difference n between the first impedance Ra and the second impedance Rb. If the first electrode 110a and the second electrode 110b are disposed on different materials, the magnification difference n between the first impedance Ra and the second impedance Rb will be large, which may easily enlarge the differential signal variation. Subsequent steps could compensate for differential signal variations caused by different materials.

Afterwards, in the step S362, as shown in the FIG. 5, the impedance analysis circuit 151 of the dynamic signal matching module 150 obtains the corrected differential signal S′ according to the first sensing signal Sa, the second sensing signal Sb and the magnification difference n.

Then, in the step S363, as shown in the FIG. 5, the signal compensation unit 153 of the dynamic signal matching module 150 obtains the calibration sequence M according to the original differential signal S and the corrected differential signal S′. As shown in the FIG. 5, in the calibration sequence M, only a part of the calibration sequence M corresponding the physiological signal has obvious amplitude, and the other parts of the calibration sequence M corresponding the noise almost have no amplitude. The calibration sequence M could be used to correctly interpret the range of the physiological signals.

Next, in step S370, as shown in the FIG. 5, the dynamic signal matching module 150 obtains the compensated calibration sequence M′ according to the calibration sequence M and the original differential signal S. The step S370 includes steps S371 to S372.

In the step S371, as shown in the FIG. 5, the signal compensation unit 153 of the dynamic signal matching module 150 obtains a first correction index point m1 and a second correction index point m2 according to the calibration sequence M. The first correction index point m1 is the starting point of the physiological signal, and the second correction index point m2 is the end point of the physiological signal. In this step, the signal compensation unit 153 of the dynamic signal matching module 150 could identify the first correction index point m1 and the second correction index point m2 according to the amplitude change of the calibration sequence M. For example, the location where the amplitude change significantly increases could be identified as the first correction index point m1, and the location where the amplitude change significantly decreases could be identified as the second correction index point m2.

Then, in the step S372, as shown in the FIG. 5, the signal compensation unit 153 of the dynamic signal matching module 150 fills the original differential signal S between the first correction index point m1 and the second correction index point m2 of the calibration sequence M to obtain the compensated calibration sequence M′. The calibration sequence M is mainly used to distinguish the physiological signals and the noise. The physiological signal may be excessively attenuated in the calibration sequence M. Therefore, the original differential signal S is needed to be filled between the first correction index point m1 and the second correction index point m2 to obtain the correct physiological signal.

Through the above steps, even if the materials of the contact points for the first electrode 110a, the second electrode 110b and the reference electrode 130 are quite different, or an insulating material is used as the contact point, measurements could be performed, and the correct physiological signals could be obtained after compensation. In this way, the first electrode 110a, the second electrode 110b and the reference electrode 130 could be arbitrarily arranged at various positions of the electronic device. Any position of the electronic device could be used to dispose the first electrode 110a, the second electrode 110b and the reference electrode 130. One-hand measurement or two-hand measurement could be achieved on the electronic device according to the user's usage habits. The following further illustrates examples in which the first electrode 110a, the second electrode 110b and the reference electrode 130 are provided in various electronic devices.

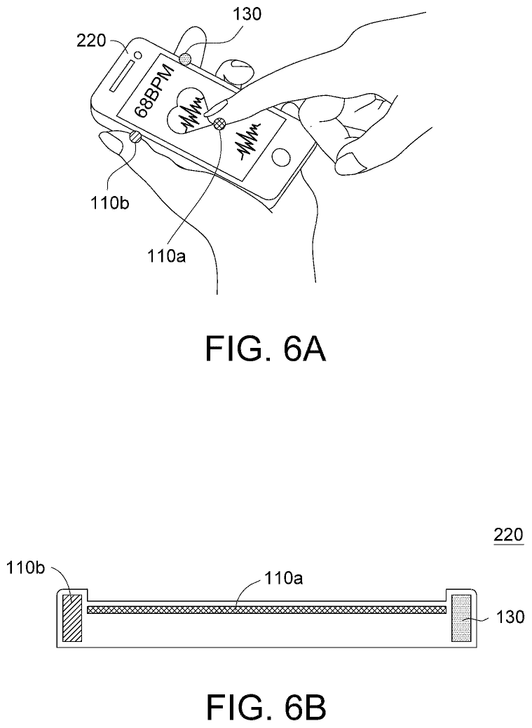

Please refer to FIG. 6A. FIG. 6A shows a schematic diagram of a mobile device 220 capable of measuring physiological signals according to an embodiment. The first electrode 110a is, for example, disposed on the screen glass plate of the mobile device 220. The second electrode 110b is, for example, disposed on one side frame of the mobile device 220. The reference electrode 130 is, for example, disposed on another side frame of the mobile device 220. The first electrode 110a, the second electrode 110b and the reference electrode 130 are isolated from each other. As shown in the FIG. 6A, the user could hold the mobile device 220 with one hand. The index finger of the user's one hand could touch the screen glass plate provided with the first electrode 110a. The thumb and the index finger of the user's another hand could respectively touch the two opposite side frames provided with the second electrode 110b and the reference electrode 130. In this way, the two-hand measurement for the physiological signals could be realized on the mobile device 220.

Please refer to FIG. 6B. FIG. 6B shows a side perspective view of the mobile device 220 of the FIG. 6A. The first electrode 110a is disposed at the inner side of the screen glass plate of the mobile device 220, and the second electrode 110b and the reference electrode 130 are respectively disposed at the inner sides of the opposite side frames of the mobile device 220. That is to say, when measuring the physiological signals, the user's fingers and the first electrode 110a, the second electrode 110b and the reference electrode 130 will be separated by the screen glass plate or the side frames. The materials of the side frames are, for example, the high-impedance plastic or the low-impedance alloy. All of the screen glass plate and the plastic side frames may have high impedance, or the impedance difference between the screen glass plate and the alloy side frames may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

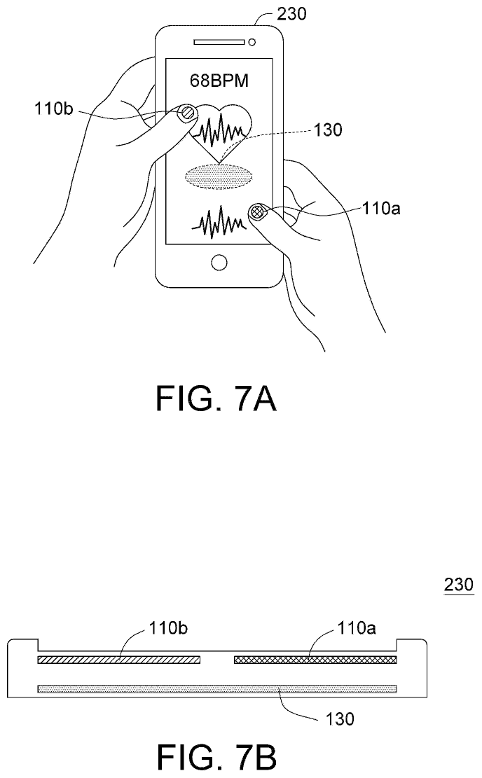

Please refer to FIG. 7A. FIG. 7A shows a schematic diagram of a mobile device 230 capable of measuring physiological signals according to another embodiment. The first electrode 110a is, for example, disposed on the right half of the screen glass plate of the mobile device 230. The second electrode 110b is, for example, disposed on the left half of the screen glass plate of the mobile device 230. The reference electrode 130 is, for example, disposed on the back plate of the mobile device 230. The first electrode 110a, the second electrode 110b and the reference electrode 130 are isolated from each other. As shown in the FIG. 7A, the user could hold the mobile device 230 with both hands. The thumb of the user's one hand could touch the right half of the screen glass plate provided with the first electrode 110a. The thumb of the user's another hand could touch the left half of the screen glass plate provided with the second electrode 110b. The user's other fingers could touch the back plate provided with the reference electrode 130. In this way, the two-hand measurement for the physiological signals could be realized on the mobile device 230.

Please refer to FIG. 7B. FIG. 7B shows a side perspective view of the mobile device 230 of the FIG. 7A. The first electrode 110a and the second electrode 110b are disposed at the inner side of the screen glass plate of the mobile device 230. The reference electrode 130 is disposed at the inner side of the back plate of the mobile device 230. That is to say, when measuring the physiological signals, the user's fingers and the first electrode 110a, the second electrode 110b and the reference electrode 130 will be separated by the screen glass plate or the side frames. The material of the back plate is, for example, the high-impedance plastic or the low-impedance alloy. Both of the screen glass plate and the plastic back plate may have high impedance, or the impedance difference between the screen glass plate and the alloy back plate may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

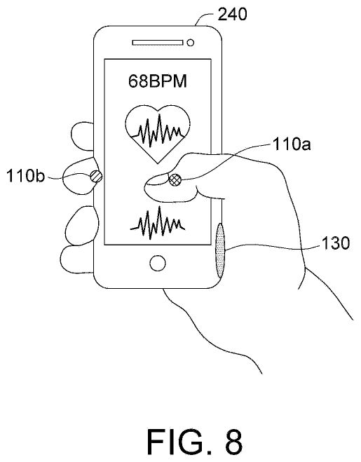

Please refer to FIG. 8. FIG. 8 shows a schematic diagram of a mobile device 240 capable of measuring physiological signals according to another embodiment. The first electrode 110a is, for example, disposed on the screen glass plate of the mobile device 240. The second electrode 110b is, for example, disposed on the upper half of the two side frames of the mobile device 240. The reference electrode 130 is, for example, disposed on the lower half of the two side frames of the mobile device 240. The first electrode 110a, the second electrode 110b and the reference electrode 130 are isolated from each other. As shown in the FIG. 8, the user could hold the mobile device 240 with one hand. The thumb of the user's one hand could touch the screen glass plate provided with the first electrode 110a. The index finger of the user's same hand could touch the upper half of the side frame provided with the second electrode 110b. The inner side of the user's palm could touch the lower half of the side frame provided with the reference electrode 130. In this way, the one-hand measurement for the physiological signals could be realized on the mobile device 240.

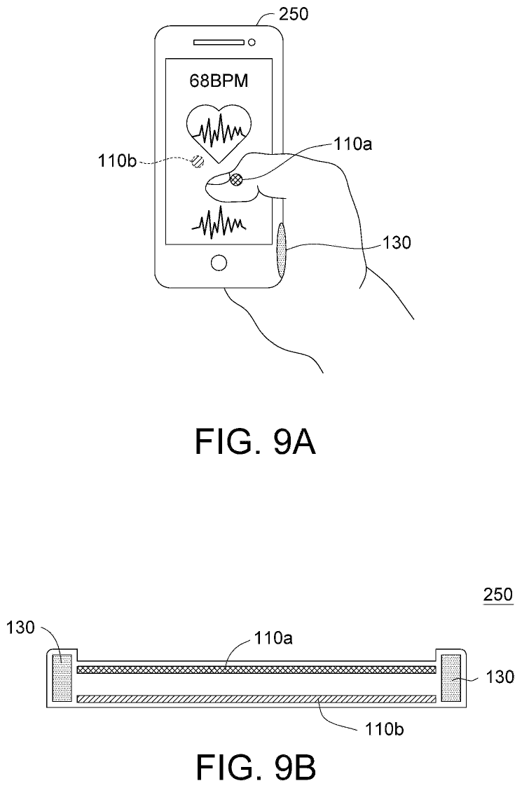

Please refer to FIG. 9A. FIG. 9A shows a schematic diagram of a mobile device 250 capable of measuring physiological signals according to another embodiment. The first electrode 110a is, for example, disposed on the screen glass plate of the mobile device 250. The second electrode 110b is, for example, disposed on the back plate of the mobile device 250. The reference electrode 130 is, for example, disposed on both of the side frames of the mobile device 250. The first electrode 110a, the second electrode 110b and the reference electrode 130 are isolated from each other. As shown in the FIG. 9A, the user could hold the mobile device 250 with one hand. The thumb of user's one hand could touch the screen glass plate provided with the first electrode 110a. The other fingers of the user's same hand could touch the back plate provided with the second electrode 110b. The inner side of the user's palm could touch the side frames provided with the reference electrode 130. In this way, the one-hand measurement for the physiological signals could be realized on the mobile device 250.

Please refer to FIG. 9B. FIG. 9B shows a side perspective view of the mobile device 250 of the FIG. 9A. The first electrode 110a is disposed at the inner side of the screen glass plate of the mobile device 250. The second electrode 110b is disposed at the inner side of the back plate of the mobile device 250. The reference electrode 130 is disposed at the inner sides of the side frames of the mobile device 250. That is to say, when measuring the physiological signals, the user's fingers or palm and the first electrode 110a, the second electrode 110b and the reference electrode 130 will be separated by the screen glass plate or the side frames. The material of the back plate is, for example, the high-impedance plastic or the low-impedance alloy. All of the screen glass plate, the plastic back plate and the plastic side frames may have high impedance, or the impedance difference between the screen glass plate, the alloy back plate and the alloy side frames may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

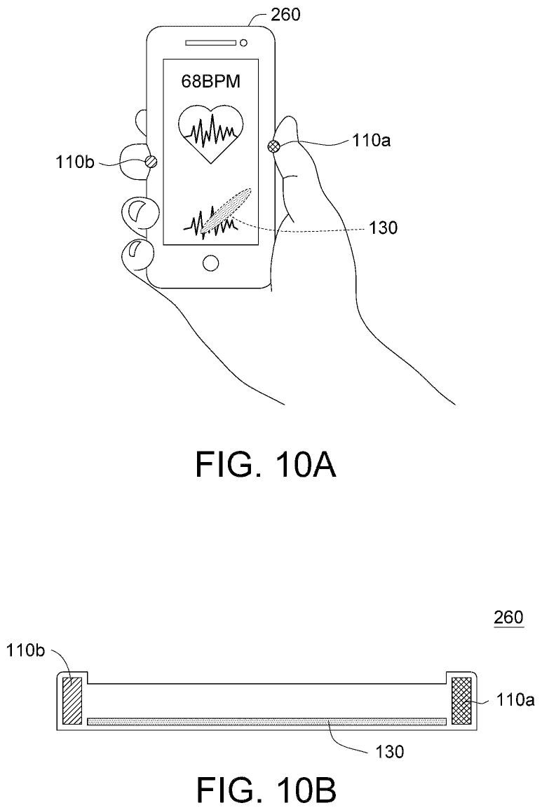

Please refer to FIG. 10A. FIG. 10A shows a schematic diagram of a mobile device 260 capable of measuring physiological signals according to another embodiment. The first electrode 110a is, for example, disposed on one side frame of the mobile device 260. The second electrode 110b is, for example, disposed on another side frame of the mobile device 260. The reference electrode 130 is, for example, disposed on the back plate of the mobile device 260. The first electrode 110a, the second electrode 110b and the reference electrode 130 are isolated from each other. As shown in the FIG. 10A, the user could hold the mobile device 260 with one hand. The thumb of the user's one hand could touch the side frame provided with the first electrode 110a. The middle finger of the user's same hand one hand could touch the side frame provided with the second electrode 110b. The inner side of the user's palm could touch the back plate provided with the reference electrode 130. In this way, the one-hand measurement for the physiological signals could be realized on the mobile device 260.

Please refer to FIG. 10B. FIG. 10B shows a side perspective view of the mobile device 260 of the FIG. 10A. The first electrode 110a is disposed at the inner side of one side frame of the mobile device 260. The second electrode 110b is disposed at the inner side of another side frame of the mobile device 260. The reference electrode 130 is disposed at the inner side of the back plate of the mobile device 260. That is to say, when measuring the physiological signals, the user's fingers or palm and the first electrode 110a, the second electrode 110b and the reference electrode 130 will be separated by the screen glass plate or the side frames. The materials of the side frame and the back plate are, for example, the high-impedance plastic or the low-impedance alloy. All of the plastic side frames and the plastic back plate may have high impedance, or the impedance difference between the alloy back plate and the plastic side frames may be too large, or the impedance difference between the plastic back plate and the alloy side frames may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

The above-mentioned embodiments of FIGS. 1A to 1B, 6A to 6B, 7A to 7B, 8, 9A to 9B, and 10A to 10B are not intended to limit the implementation of this technology for the mobile device. For example, the first electrode 110a could be disposed on the screen glass plate, the side frames or the back plate of the mobile device. The second electrode 110b could be disposed on the screen glass plate, the side frames or the back plate of the mobile device. The reference electrode 130 could be disposed on the screen glass plate, the side frames or the back plate of the mobile device. Various implementations could use the above-mentioned impedance front-end circuit technology and the dynamic signal matching technology to perform measurements and obtain the correct physiological signals after compensation.

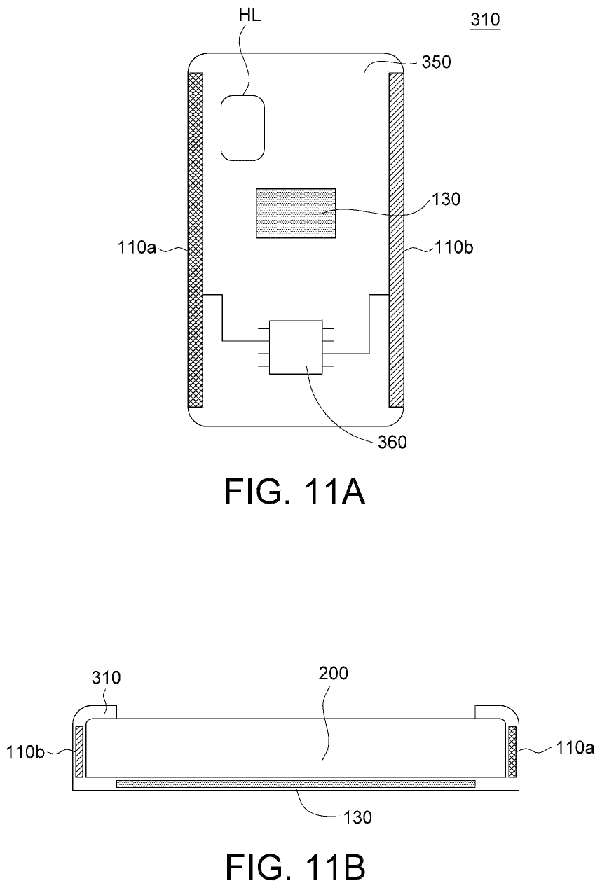

Please refer to FIG. 11A. FIG. 11A shows a schematic diagram of a mobile device protective case 310 capable of measuring physiological signals according to another embodiment. The mobile device protective case 310 includes a case 350, the first electrode 110a, the second electrode 110b, the reference electrode 130 and a processing system 360. The case 350 had a lens hole HL. The first electrode 110a is disposed at the inner side of the case 350. The second electrode 110b is disposed at the inner side of the case 350. The reference electrode 130 is disposed at the inner side of the case 350. The processing system 360 is disposed at the inner side of the case 350. The processing system 360 is coupled to the first electrode 110a, the second electrode 110b and the reference electrode 130. The processing system 360 includes the impedance front-end circuit module 140 and the dynamic signal matching module 150. The processing system 360 is, for example, a chip, a circuit or a circuit board. The first electrode 110a is, for example, disposed on one side frame of the case 350 of the mobile device protective case 310. The second electrode 110b is, for example, disposed on another side frame of the case 350 of the mobile device protective case 310. The reference electrode 130 is, for example, disposed on the back plate of the case 350 of the mobile device protective case 310. The first electrode 110a, the second electrode 110b and the reference electrode 130 are isolated from each other.

Please refer to FIG. 11B. FIG. 11B shows a side perspective view of the mobile device protective case 310 of the FIG. 11A and the mobile device 200. The first electrode 110a is disposed at the inner side of one side frame of the case 350 of the mobile device protective case 310. The second electrode 110b is disposed at the inner side of another side frame of the case 350 of the mobile device protective case 310. The reference electrode 130 is disposed at the inner side of the back plate of the case 350 of the mobile device protective case 310. The mobile device 200 could be covered by the mobile device protective case 310. The material of the case 350 of the mobile device protective case 310 is, for example, the high-impedance plastic or the low-impedance alloy. The plastic case 350 of the mobile device protective case 310 may have high impedance, or the impedance difference between the plastic part of the case 350 and the alloy part of the case 350 may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

The above embodiments in FIGS. 11A to 11B are not intended to limit the implementation of this technology for the mobile device protective case. For example, the first electrode 110a, the second electrode 110b and the reference electrode 130 could be disposed at any location of the case of the mobile device protective case. Various implementations could use the above-mentioned impedance front-end circuit technology and the dynamic signal matching technology to perform measurements and obtain the correct physiological signals after compensation.

Please refer to FIG. 12A. FIG. 12A shows a schematic diagram of a laptop 410 capable of measuring physiological signals according to an embodiment. The first electrode 110a is, for example, disposed on the right half of the screen glass plate of the laptop 410. The second electrode 110b is, for example, disposed on the left half of the screen glass plate of the laptop 410. The reference electrode 130 is, for example, disposed on the screen back plate of the laptop 410. The first electrode 110a, the second electrode 110b and the reference electrode 130 are isolated from each other. As shown in the FIG. 12A, the user could hold the laptop 410 with both hands. The thumb of the user's one hand could touch the right half of the screen glass plate provided with the first electrode 110a. The thumb of the user's another hand could touch left half of the screen glass plate provided with the second electrode 110b. The user's other fingers could touch the screen back plate provided with the reference electrode 130. In this way, the two-hand measurement for the physiological signals could be realized on the laptop 410.

Please refer to FIG. 12B. FIG. 12B shows a side perspective view of the laptop 410 of the FIG. 12A. The first electrode 110a and the second electrode 110b are disposed at the inner side of the screen glass plate of the laptop 410. The reference electrode 130 is disposed at the inner side of the screen back plate of the laptop 410. That is to say, when measuring the physiological signals, the user's fingers and the first electrode 110a, the second electrode 110b and the reference electrode 130 will be separated by the screen glass plate or the screen back plate. The material of the screen back plate is, for example, the high-impedance plastic or the low-impedance alloy. Both of the screen glass plate and the plastic screen back plate may have high impedance, or the impedance difference between the screen glass plate and the alloy back plate may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

The above embodiments in the FIGS. 12A to 12B are not intended to limit the implementation of this technology for the laptop. For example, the first electrode 110a could be disposed on the screen glass plate, the screen back plate or the screen side frame. The second electrode 110b could be disposed on the screen glass plate, the screen back plate or the screen side frame. The reference electrode 130 could be disposed on the screen glass plate, the screen back plate or the screen side frame. Various implementations could use the above-mentioned impedance front-end circuit technology and the dynamic signal matching technology to perform measurements and obtain the correct physiological signals after compensation.

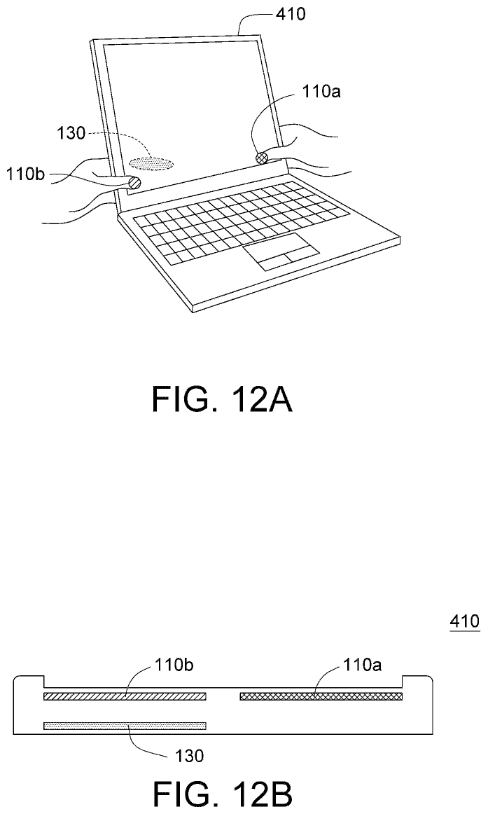

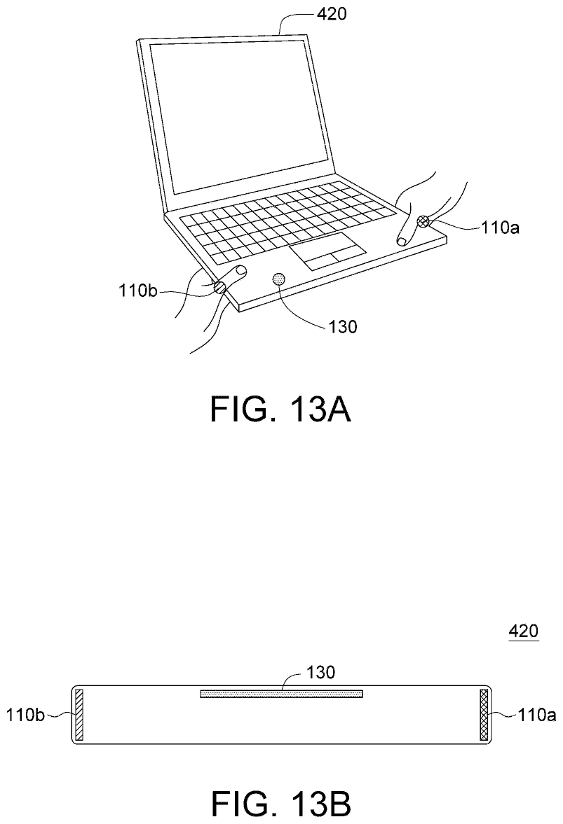

Please refer to FIG. 13A. FIG. 13A shows a schematic diagram of a laptop 420 capable of measuring physiological signals according to another embodiment. The first electrode 110a is, for example, disposed on one host side frame of the laptop 420. The second electrode 110b is, for example, disposed on another host side frame of the laptop 420. The reference electrode 130 is, for example, disposed on the host front plate of the laptop 420. The first electrode 110a, the second electrode 110b and the reference electrode 130 are isolated from each other. As shown in the FIG. 13A, the user could hold the laptop 410 with both hands. The thumb of the user's one hand could touch the host side frame provided with the first electrode 110a. The thumb of the user's another hand could touch the host side frame provided with the second electrode 110b. The user's other fingers could touch the host front plate provided with the reference electrode 130. In this way, the two-hand measurement for the physiological signals could be realized on the laptop 420.

Please refer to FIG. 13B. FIG. 13B shows a side perspective view of the laptop 420 of the FIG. 13A. The first electrode 110a and the second electrode 110b are disposed at the inner sides of the host side frames of the laptop 420. The reference electrode 130 is disposed at the inner side of the host front plate of the laptop 420. That is to say, when measuring the physiological signals, the user's fingers and the first electrode 110a, the second electrode 110b and the reference electrode 130 will be separated by the host side frames and the host front plate. The materials of the host side frames and the host front plate are, for example, the high-impedance plastic or the low-impedance alloy. All of the host side frames and the host front plate may have high impedance, or the impedance difference between the host side frames and the host front plate may be too large. Once a high-impedance material is used as a contact point, or the impedance difference between the materials used for the contact points is too large, the coupling capacitance effect may not be produced and the differential signal variation becomes too large. This embodiment uses the impedance front-end circuit technology and the dynamic signal matching technology to perform the measurements, and could obtain the correct physiological signals after compensation.

The above embodiments in FIGS. 13A to 13B are not intended to limit the implementation of this technology for the laptop. For example, the first electrode 110a could be disposed on the host back plate, the host front plate or the host side frames of the laptop. The second electrode 110b could be disposed on the host back plate, the host front plate or the host side frames of the laptop. The reference electrode 130 could be disposed on the host back plate, the host front plate or the host side frames of the laptop. Various implementations could use the above-mentioned impedance front-end circuit technology and the dynamic signal matching technology to perform measurements and obtain the correct physiological signals after compensation.

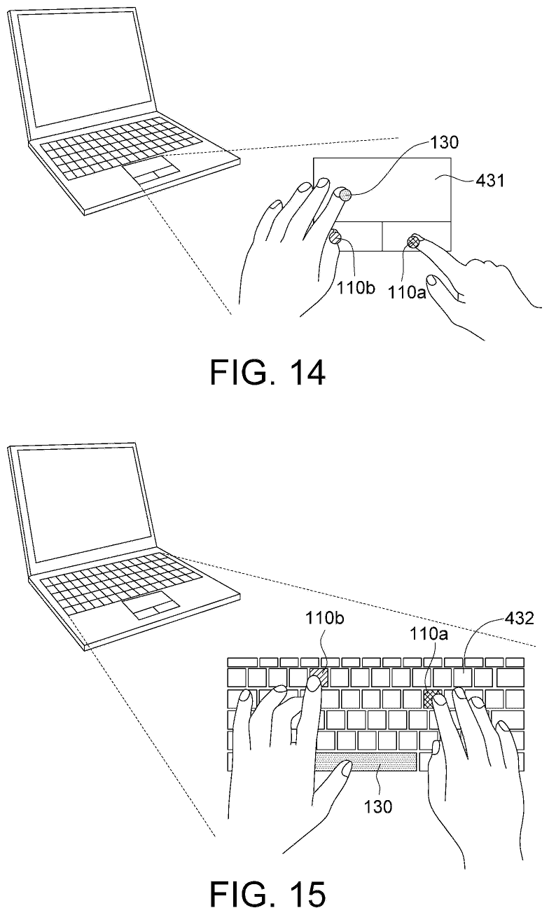

Please refer to FIG. 14. FIG. 14 shows a schematic diagram of a touchpad 431 capable of measuring physiological signals according to an embodiment. The first electrode 110a is, for example, disposed on the touchpad right button of the touchpad 431. The second electrode 110b is, for example, disposed on the touchpad left button of the touchpad 431. The reference electrode 130 is, for example, disposed on the touchpad main area of the touchpad 431. The first electrode 110a, the second electrode 110b and the reference electrode 130 are isolated from each other. As shown in the FIG. 14, the user could touch the touchpad with both hands. The thumb and the index finger of user's one hand could touch the touchpad left button and the touchpad main area provided with the second electrode 110b and the reference electrode 130 respectively. The index finger of the user's another hand could touch the touchpad right button provided with the first electrode 110a. In this way, the two-hand measurement for the physiological signals could be realized on the touchpad 431.

The above-mentioned embodiment in FIG. 14 is not intended to limit the implementation of this technology for the touchpad. For example, the first electrode 110a could be disposed on the touchpad main area, the touchpad right button or the touchpad left button. The second electrode 110b could be disposed on the touchpad main area, the touchpad right button or the touchpad left button. The reference electrode 130 could be disposed on the touchpad main area, the touchpad right button or the touchpad left button. Various implementations could use the above-mentioned impedance front-end circuit technology and the dynamic signal matching technology to perform measurements and obtain the correct physiological signals after compensation.

Please refer to FIG. 15. FIG. 15 shows a schematic diagram of a keyboard 432 capable of measuring physiological signals according to an embodiment. The first electrode 110a is, for example, disposed on a first button of the keyboard 432. The second electrode 110b is, for example, disposed on a second button of the keyboard 432. The reference electrode 130 is, for example, disposed on a third button of the keyboard 432. The first electrode 110a, the second electrode 110b and the reference electrode 130 are isolated from each other. As shown in the FIG. 15, the user could touch the keyboard 432 with both hands. The user's fingers could respectively touch three buttons provided with the first electrode 110a, the second electrode 110b and the reference electrode 130. In this way, the two-hand measurement for the physiological signals could be realized on the keyboard 432.

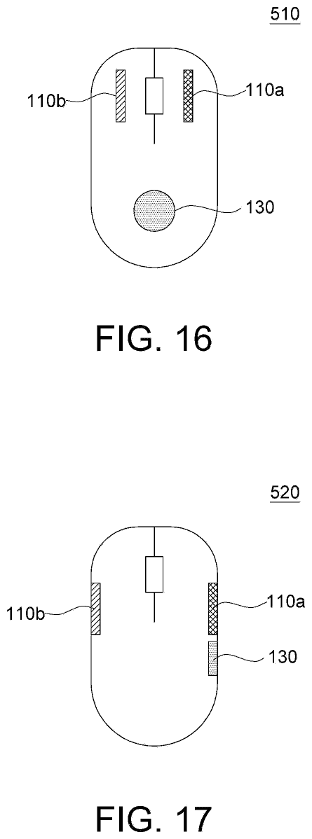

Please refer to FIG. 16. FIG. 16 shows a schematic diagram of a mouse 510 capable of measuring physiological signals according to an embodiment. The first electrode 110a is, for example, disposed on the right button of the mouse 510. The second electrode 110b is, for example, disposed on the left button of the mouse 510. The reference electrode 130 is, for example, disposed on the body of the mouse 510. The first electrode 110a, the second electrode 110b and the reference electrode 130 are isolated from each other. The user could hold the mouse 510 with one hand. The index finger of the user's one hand could touch the left button of the mouse 510 provided with the second electrode 110b. The middle finger of the user's same hand could touch the right button of the mouse 510 provided with the first electrode 110a. The inner side of the palm of the user's same hand could touch the body of the mouse 510 provided with the reference electrode 130. In this way, the one-hand measurement for the physiological signals could be realized on the mouse 510.

The above-mentioned embodiment of the FIG. 16 is not intended to limit the implementation of this technology for the mouse. For example, the first electrode 110a could be disposed on the left button, the right button or the body of the mouse. The second electrode 110b could be disposed on the left button, the right button or the body of the mouse. The reference electrode 130 could be disposed on the left button, the right button or the body of the mouse. Various implementations could use the above-mentioned impedance front-end circuit technology and the dynamic signal matching technology to perform measurements and obtain the correct physiological signals after compensation.

Please refer to FIG. 17. FIG. 17 shows a schematic diagram of a mouse 520 capable of measuring physiological signals according to another embodiment. The first electrode 110a is, for example, disposed on the upper half of the right case of the mouse 520. The second electrode 110b is, for example, disposed on the left case of the mouse 510. The reference electrode 130 is, for example, disposed on the lower half of the right case of the mouse 520. The first electrode 110a, the second electrode 110b and the reference electrode 130 are isolated from each other. The user could hold the mouse 520 with one hand. The thumb of the user's one hand could touch the left case of the mouse 520 provided with the second electrode 110b. The little finger of the user's same hand could touch the upper half of the right case of the mouse 520 provided with the first electrode 110a. The inner side of the palm of the user's same hand could touch the lower half of the right case of the mouse 520 provided with the reference electrode 130. In this way, the one-hand measurement for the physiological signals could be realized on the mouse 520.

The above-mentioned embodiment of the FIG. 17 is not intended to limit the implementation of this technology for the mouse. For example, the first electrode 110a could be disposed on the left case or the right case of the mouse. The second electrode 110b could be disposed on the left case or the right case of the mouse. The reference electrode 130 could be disposed on the left case or the right case of the mouse. Various implementations could use the above-mentioned impedance front-end circuit technology and the dynamic signal matching technology to perform measurements and obtain the correct physiological signals after compensation.

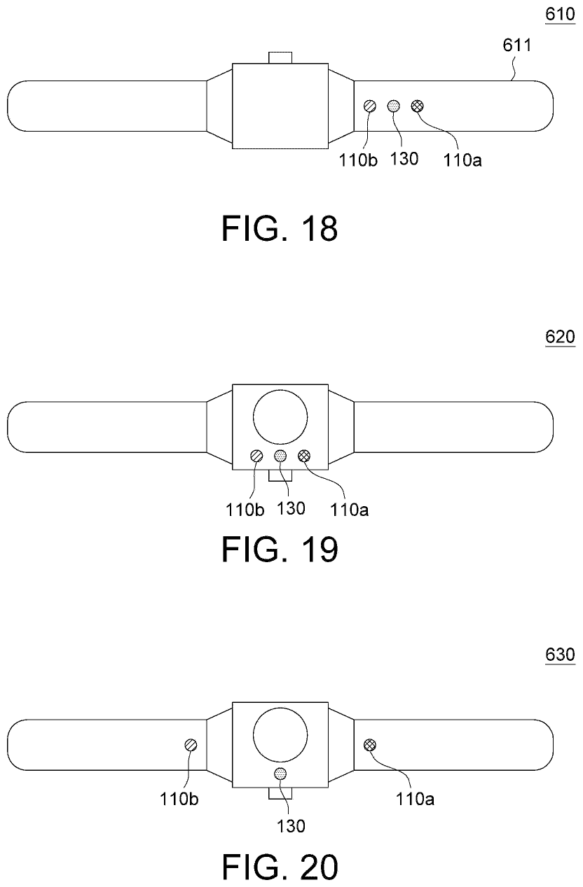

Please refer to FIG. 18. FIG. 18 shows a schematic diagram of a smart watch 610 capable of measuring physiological signals according to an embodiment. The first electrode 110a, the second electrode 110b and the reference electrode 130 are disposed on a watch strap 611 of the smart watch 610. The first electrode 110a, the second electrode 110b and the reference electrode 130 are isolated from each other. The user could touch the watch strap 611 with one hand. In this way, the one-hand measurement for the physiological signals could be realized on the smart watch 610.

Please refer to FIG. 19. FIG. 19 shows a schematic diagram of a smart watch 620 capable of measuring physiological signals according to another embodiment. The first electrode 110a, the second electrode 110b and the reference electrode 130 are disposed on the body of the smart watch 620. The first electrode 110a, the second electrode 110b and the reference electrode 130 are isolated from each other. The user could touch the smart watch 620 with one hand. In this way, the one-hand measurement for the physiological signals could be realized on the smart watch 620.

Please refer to FIG. 20. FIG. 20 shows a schematic diagram of a smart watch 630 capable of measuring physiological signals according to another embodiment. The first electrode 110a is disposed on one watch strap of the smart watch 630. The second electrode 110b is disposed on another watch strap of the smart watch 630. The reference electrode 130 is disposed on the body of the smart watch 630. The first electrode 110a, the second electrode 110b and the reference electrode 130 are isolated from each other. The user could touch the body and the watch strap of the smart watch 630 with one hand. In this way, the one-hand measurement for the physiological signals could be realized on the smart watch 630.

The above-mentioned embodiments of the FIGS. 18 to 20 are not intended to limit the implementation of this technology for the smart watch. For example, the first electrode 110a could be disposed on the watch straps or the body of the smart watch. The second electrode 110b could be disposed on the watch straps or the body of the smart watch. The reference electrode 130 could be disposed on the watch straps or the body of the smart watch. Various implementations could use the above-mentioned impedance front-end circuit technology and the dynamic signal matching technology to perform measurements and obtain the correct physiological signals after compensation.

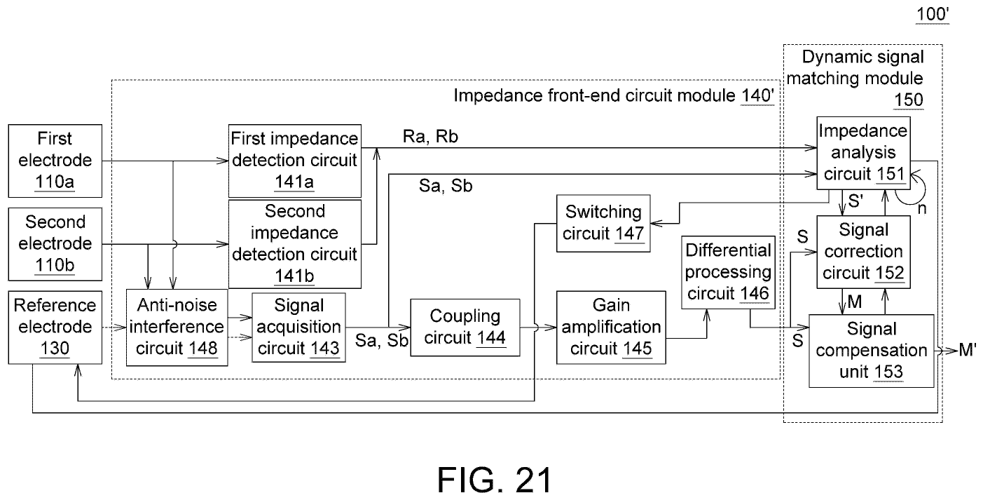

Please refer to FIG. 21. FIG. 21 shows a block diagram of a physiological signal measurement system 100′ according to another embodiment. In another embodiment, the physiological signal measurement system 100′ could also be applied to a vehicle. An impedance front-end circuit module 140′ of the physiological signal measurement system 100′ further includes an anti-noise interference circuit 148. The anti-noise interference circuit 148 is used to filter noise generated by vibration for improving the measurement accuracy for the physiological signals. For example, the physiological signal measurement system 100′ could be applied to a steering wheel, a seat, or a gear lever.

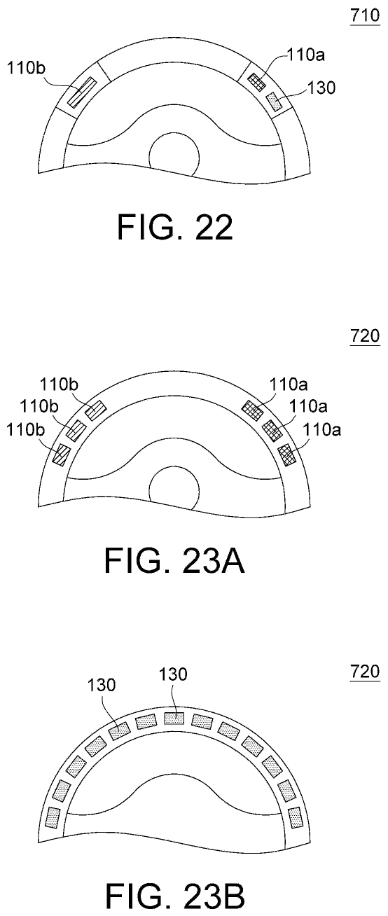

Please refer to FIG. 22. FIG. 22 shows a schematic diagram of a steering wheel 710 capable of measuring physiological signals according to an embodiment. The first electrode 110a, the second electrode 110b and the reference electrode 130 are disposed on the front side of the steering wheel 710. The first electrode 110a, the second electrode 110b and the reference electrode 130 are isolated from each other. The user could touch the steering wheel 710 with both hands. In this way, the two-hand measurement for the physiological signals could be realized on the steering wheel 710.

Please refer to FIGS. 23A to 23B. FIG. 23A illustrates a front view of a steering wheel 720 capable of measuring physiological signals according to another embodiment. FIG. 23B illustrates a back view of the steering wheel 720 capable of measuring physiological signals according to another embodiment. The first electrode 110a and the second electrode 110b are disposed on the front side of the steering wheel 720. The reference electrode 130 is disposed on the back side of the steering wheel 720. The first electrode 110a, the second electrode 110b and the reference electrode 130 are isolated from each other. The user could hold the steering wheel 720 with both hands. In this way, the two-hand measurement for the physiological signals could be realized on the steering wheel 720.

The above-mentioned embodiments of the FIGS. 22 to 23 are not intended to limit the implementation of this technology for the steering wheel. For example, the first electrode 110a could be disposed on the front side or the back side of the steering wheel. The second electrode 110b could be disposed on the front side or the back side of the steering wheel. The reference electrode 130 could be disposed on the front side or the back side of the steering wheel. Various implementations could use the above-mentioned impedance front-end circuit technology and the dynamic signal matching technology to perform measurements and obtain the correct physiological signals after compensation.

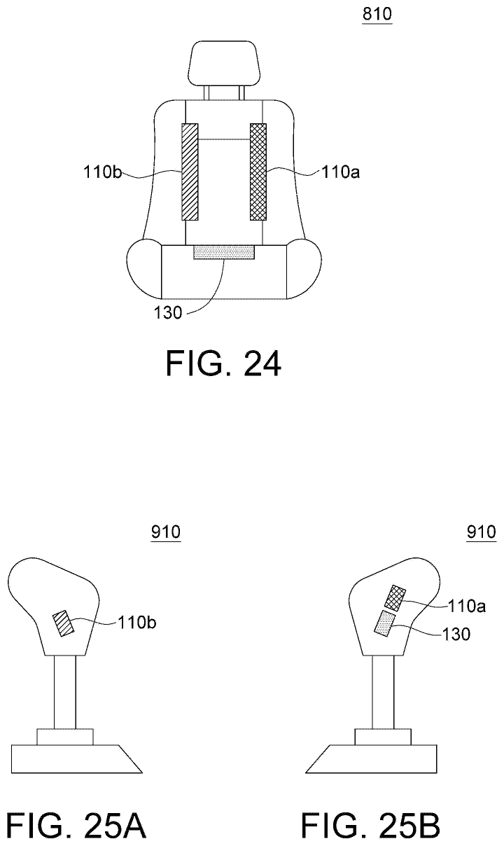

Please refer to FIG. 24. FIG. 24 illustrates a schematic diagram of a seat 810 capable of measuring physiological signals according to an embodiment. The first electrode 110a and the second electrode 110b are disposed on the chair back of the seat 810. The reference electrode 130 is disposed on the chair base of the seat 810. The first electrode 110a, the second electrode 110b and the reference electrode 130 are isolated from each other. The user could touch the seat 810 with user's body. In this way, the measurement for the physiological signals could be realized on the seat 810.

The above-mentioned embodiment of the FIG. 24 is not intended to limit the implementation of this technology for the seat. For example, the first electrode 110a could be disposed on the chair back or the chair base of the seat. The second electrode 110b could be disposed on the chair back or the chair base of the seat. The reference electrode 130 could be disposed on the chair back or the chair base of the seat. Various implementations could use the above-mentioned impedance front-end circuit technology and the dynamic signal matching technology to perform measurements and obtain the correct physiological signals after compensation.

Please refer to FIGS. 25A to 25B. FIG. 25A shows a schematic diagram of the left side of a gear lever 910 capable of measuring physiological signals according to an embodiment. FIG. 25B shows a schematic diagram of the right side of the gear lever 910 capable of measuring physiological signals according to an embodiment. The first electrode 110a and the reference electrode 130 are disposed on the right side surface of the gear lever 910. The second electrode 110b is disposed on the left side surface of the gear lever 910. The first electrode 110a, the second electrode 110b and the reference electrode 130 are isolated from each other. The user could touch the gear lever 910 with one hand. In this way, the one-hand measurement for the physiological signals could be realized on the gear lever 910.

The above-mentioned embodiments of the FIGS. 25A to 25B are not intended to limit the implementation of this technology for the gear lever. For example, the first electrode 110a could be disposed on the left side surface or the right side surface of the gear lever. The second electrode 110b could be disposed on the left side surface or the right side surface of the gear lever. The reference electrode 130 could be disposed on the left side surface or the right side surface of the gear lever. Various implementations could use the above-mentioned impedance front-end circuit technology and the dynamic signal matching technology to perform measurements and obtain the correct physiological signals after compensation.

According to the above embodiments, the impedance front-end circuit technology and the dynamic signal matching technology are used. Even if the materials of the contact points of the first electrode 110a, the second electrode 110b and the reference electrode 130 are quite different, or an insulating material is used as the contact point, the measurements could be performed, and the correct physiological signals could be obtained after compensation. In this way, the first electrode 110a, the second electrode 110b and the reference electrode 130 could be arranged at various positions of the electronic device. Any position of the electronic device could be used to dispose the first electrode 110a, the second electrode 110b and the reference electrode 130. The one-hand measurement or the two-hand measurement could be achieved on the electronic device according to the user's usage habits.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A physiological signal measurement system, comprising:
a first electrode;
a second electrode;
a reference electrode, wherein the first electrode and the reference electrode are used to obtain a first sensing signal, and the second electrode and the reference electrode are used to obtain a second sensing signal;
an impedance front-end circuit module, configured to detect a first impedance of the first electrode and a second impedance of the second electrode, and obtain an original differential signal according to the first sensing signal and the second sensing signal; and
a dynamic signal matching module, configured to obtain a calibration sequence according to the first impedance, the second impedance and the original differential signal, and obtain a compensated calibration sequence according to the calibration sequence and the original differential signal.

2. The physiological signal measurement system according to claim 1, wherein the impedance front-end circuit module comprises:
a signal acquisition circuit, connected the first electrode, the second electrode and the reference electrode, wherein the signal acquisition circuit is used to acquire the first sensing signal and the second sensing signal;
a coupling circuit, connected the signal acquisition circuit, wherein the coupling circuit is used to improve a signal-to-noise ratio of the first sensing signal and the second sensing signal;
a gain amplification circuit, connected to the coupling circuit, wherein the gain amplification circuit is used to amplify the first sensing signal and the second sensing signal; and
a differential processing circuit, connected to the gain amplification circuit, wherein the differential processing circuit obtains an original differential signal according to the first sensing signal and the second sensing signal.

3. The physiological signal measurement system according to claim 2, wherein the coupling circuit comprises:
a first negative impedance circuit, connected to the first electrode;
a first high impedance circuit, connected to the first negative impedance circuit;
a second negative impedance circuit, connected to the second electrode; and
a second high impedance circuit, connected to the second negative impedance circuit.

4. The physiological signal measurement system according to claim 1, wherein the dynamic signal matching module comprises:
an impedance analysis circuit, configured to analyze a magnification difference between the first impedance and the second impedance, and obtain a corrected differential signal according to the first sensing signal, the second sensing signal and the magnification difference;
a signal correction circuit, configured to obtain the calibration sequence according to the original differential signal and the corrected differential signal; and
a signal compensation unit, configured to obtain a first correction index point and a second correction index point according to the calibration sequence, and fill the original differential signal between the first correction index point and the second correction index point of the calibration sequence to obtain the compensated calibration sequence.

5. The physiological signal measurement system according to claim 1, wherein first electrode is disposed on a screen glass plate, a side frame or a back plate of a mobile device; the second electrode is disposed on the screen glass plate, the side frame or the back plate of the mobile device, and the reference electrode is disposed on the screen glass plate, the side frame or the back plate of the mobile device.

6. The physiological signal measurement system according to claim 1, wherein the first electrode, the second electrode and reference electrode are isolated from each other.

7. The physiological signal measurement system according to claim 1, wherein first electrode is disposed on a screen glass plate, a back plate or a side frame of a laptop; the second electrode is disposed on the screen glass plate, the back plate or the side frame of the laptop; and the reference electrode is disposed on the screen glass plate, the back plate or the side frame of the laptop.

8. The physiological signal measurement system according to claim 1, wherein the first electrode is disposed on a host back plate, a host front plate or a host side frame of a laptop; the second electrode is disposed on the host back plate, the host front plate or the host side frame of the laptop; and the reference electrode is disposed on the host back plate, the host front plate or the host side frame of the laptop.

9. The physiological signal measurement system according to claim 1, wherein the first electrode is disposed on a touchpad main area, a touchpad left button or a touchpad right button of a touchpad; the second electrode is disposed on the touchpad main area, the touchpad left button or the touchpad right button of the touchpad; and the reference electrode is disposed on the touchpad main area, the touchpad left button or the touchpad right button of the touchpad.

10. The physiological signal measurement system according to claim 1, wherein the first electrode is disposed on a first button of a keyboard, the second electrode is disposed on a second button of the keyboard, and the reference electrode is disposed on a third button of the keyboard.

11. The physiological signal measurement system according to claim 1, wherein the first electrode is disposed on a left button, a right button or a main body of a mouse, the second electrode is disposed on the left button, the right button or the main body of the mouse, and the reference electrode is disposed on the left button, the right button or the main body of the mouse.

12. The physiological signal measurement system according to claim 1, wherein the first electrode is disposed on a left case or a right case of a mouse, the second electrode the left case or the right case of the mouse, and the reference electrode the left case or the right case of the mouse.

13. The physiological signal measurement system according to claim 1, wherein the first electrode, the second electrode and the reference electrode are disposed on a watch strap of a smart watch.

14. The physiological signal measurement system according to claim 1, wherein the first electrode, the second electrode and the reference electrode are disposed on a watch body of a smart watch.

15. The physiological signal measurement system according to claim 1, wherein, the first electrode is disposed on a watch body or a watch strap of a smart watch, the second electrode is disposed on the watch body or the watch strap of the smart watch, and the reference electrode is disposed on the watch body or the watch strap of the smart watch.

16. The physiological signal measurement system according to claim 1, wherein the first electrode, the second electrode and the reference electrode are disposed at a front side of a steering wheel.

17. The physiological signal measurement system according to claim 1, wherein the first electrode is disposed at a front side or a back side of a steering wheel, the second electrode is disposed at the front side or the back side of the steering wheel, and the reference electrode is disposed at the front side or the back side of the steering wheel.

18. The physiological signal measurement system according to claim 1, wherein the first electrode is disposed on a chair back or a chair base of a seat, the second electrode is disposed on the chair back or the chair base of the seat, and the reference electrode is disposed on the chair back or the chair base of the seat.

19. The physiological signal measurement system according to claim 1, wherein the first electrode is disposed on a left side surface or a right side surface of a gear lever, the second electrode is disposed on the left side surface or the right side surface of the gear lever, and the reference electrode is disposed on the left side surface or the right side surface of the gear lever.

20. A physiological signal measurement method, comprising:
detecting a first impedance of a first electrode;
detecting a second impedance of a second electrode;
obtaining a first sensing signal;
obtaining a second sensing signal;
obtaining an original differential signal according to the first sensing signal and the second sensing signal;
obtaining a calibration sequence according to the first impedance, the second impedance and the original differential signal; and
obtaining a compensated calibration sequence according to the calibration sequence and the original differential signal.

21. The physiological signal measurement method according to claim 20, wherein the step of obtaining the calibration sequence according to the first impedance, the second impedance and the original differential signal includes:
analyzing a magnification difference between the first impedance and the second impedance;
obtaining a corrected differential signal according to the first sensing signal, the second sensing signal and the magnification difference; and
obtaining the calibration sequence according to the original differential signal and the corrected differential signal.

22. The physiological signal measurement method according to claim 20, wherein the step of obtaining the compensated calibration sequence according to the calibration sequence and the original differential signal includes:
obtaining a first correction index point and a second correction index point according to the calibration sequence; and
filling the original differential signal between the first correction index point and the second correction index point of the calibration sequence to obtain the compensated calibration sequence.

23. A mobile device protective case, comprising:
a case;
a first electrode, disposed at an inner side of the case;
a second electrode, disposed at the inner side of the case;
a reference electrode, disposed at the inner side of the case, wherein the first electrode and the reference electrode are used to obtain a first sensing signal, and the second electrode and the reference electrode are used to obtain a second sensing signal; and
a processing system, disposed at the inner side of the case; wherein the processing system is coupled to the first electrode, the second electrode and the reference electrode, and the processing system comprises:
an impedance front-end circuit module, configured to detect a first impedance of the first electrode and a second impedance of the second electrode, and obtain an original differential signal according to the first sensing signal and the second sensing signal; and
a dynamic signal matching module, configured to obtain a calibration sequence according to the first impedance, the second impedance and the original differential signal, and obtain a compensated calibration sequence according to the calibration sequence and the original differential signal.
